(19) United States
(12) Patent Application Publication
(10) Pub. No.: US 2007/0112269 A1
(43) Pub. Date: May 17, 2007

(54) Clutter filtering with small ensemble lengths in ultrasound imaging

(75) Inventors: Laurence Germond-Rouet, Paris (FR); Thanasis Loupas, Greece (GR); Odile Bonnefous, Rueil-Malmaison (FR)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 10/573,064

(22) PCT Filed: Sep. 2, 2004
(86) PCT No.: PCT/IB04/02892

§ 371 Date: Mar. 23, 2006

(30) Foreign Application Priority Data
Sep. 30, 2003 (EP) 03300139.7

(51) Int. Cl. A61B 8/00

(52) U.S. Cl. 600/443

(57) ABSTRACT

An ultrasound imaging system comprising: a probe (10) with an array of transducer elements (12) for acquiring ultrasound data of a body, including moving tissue and fluid flow; a beamforming system (10, 12, 14, 16) for emitting and receiving ultrasound beams in said body, which uses, for each transmission beam, an ensemble length of more than two temporal samples and less than eight; processing means (40, 50) to process flow Doppler signals comprising adaptive clutter demodulation (21) applied on amplitude signals and mean clutter demodulation applied on phase signals followed by high-pass filtering (22, 32); and display means (55,70) to display images based on said processed flow Doppler signals.

### FIELD OF THE INVENTION

The invention relates to an ultrasound, phased array imaging system and more particularly, to an imaging system having means to form either 2-D or 3-D motion images of moving parts of a body. These moving parts are typically blood flows in vessels such as arteries or the heart. A tissue is defined as clutter.

The invention particularly finds applications in the field of medical ultrasound imaging.

### BACKGROUND OF THE INVENTION

Clutter filtering is necessary to extract flow information from received Doppler signals. In current systems, the number of Doppler temporal signal samples used to produce flow information is usually between eight and sixteen. The number of temporal signal samples, i. e. successive signals along a transmission beam, is defined as an “ensemble length”.

Ultrasonic images are subject to image artifacts arising from a number of sources such as reverberation, multipath echoes, and coherent wave interference. These artifacts manifest themselves in various ways in the images, which can be broadly described as tissue image. Strong anatomic structures like arterial walls or cardiac walls mask the weak signals generated by blood. Accordingly, it would be desirable to provide ultrasonic image information in a format in which tissue structures, called clutter, does not significantly impair the images of the body region. For example, it would be desirable to provide ultrasonic image information in a format in which tissue information may be filtered for rejection from flow information.

It is already known to image the body using Doppler information. Doppler information has been used to image the body in two distinct ways. One Doppler imaging technique is commonly referred to as Doppler velocity imaging. As is well known, this technique involves the acquisition of Doppler data at different locations called sample volumes over the image plane of an ultrasonic image. The Doppler data is acquired over time and used to estimate the Doppler phase shift or frequency at each discrete sample volume. The Doppler phase shift or frequency corresponds to the velocity of tissue motion or fluid flow within the body, with the polarity of the shift indicating direction of motion or flow. This information may be color coded in accordance with the magnitude of the shift or velocity and of its polarity, and usually overlaid over a structural image of the tissue in the image plane to define the structure of the moving organs or flowing fluids. The colors in the image can provide an indication of the speed of blood flow and its direction in the heart and blood vessels, for instance.

A second Doppler technique is known as power Doppler. This power Doppler technique does not provide estimations of the velocity of motion of organ or of fluid flow. Instead, this power Doppler technique provides the measured signal intensity of the received Doppler signals that exhibit a Doppler shift. This Doppler signal intensity can be measured at each sample volume and displayed in a color variation. Unlike Doppler velocity imaging, power Doppler does not present the problems of directionality determination and low sensitivity that are characteristic of velocity imaging. Color power Doppler simply displays the Doppler signal intensity at a sample volume in a coded color.

Like color Doppler velocity imaging, the color power Doppler display is conventionally displayed with a structural B mode image to define the organ or tissue structure in which motion is occurring. Since the value at each sample volume can be averaged over time or based upon a peak value, and is not subject to the constant changes of velocity and direction which are characteristic of Doppler velocity signals, the color power Doppler display can be presented as a stable display of motion or flow conditions in the body.

It is already known from the publication entitled “Clutter Filters Adapted to Tissue Motion in Ultrasound Color Flow Imaging”, by S. Bjaerum, H. Torp, K. Kristffersen, in IEEE Transactions on Ultrasonics, Ferroelectrics and Frequency Control, Vol. 49, no. 6, pp. 693-704, June 2002, to process ultrasonic image data for clutter filtering. The ultrasonic image data are usually produced by a number of Doppler signal samples from eight to sixteen ensemble lengths (column 1, end of the first paragraph). Clutter rejection is performed by mixing down the signal with the estimate of the mean frequency prior to high pass filtering. The best results were obtained by mixing down the signal with non-constant phase increments estimated from the signal. This constitutes an adaptive clutter filtering algorithm for color flow velocity imaging.

### SUMMARY OF THE INVENTION

It is an object of the invention to provide an ultrasound imaging system comprising processing means to increase the frame rate of Doppler flow velocity imaging or Doppler power imaging by reducing the number of temporal signal samples, called ensemble length EL, used to produce Doppler information. It is particularly an object of the invention to produce Doppler information using a number of ensemble lengths inferior to or equal to six. Preferably, it is an object of the invention to provide an ultrasound imaging system comprising processing means to use a number of ensemble lengths, which is reduced to three or four ensemble lengths, out of the eight to sixteen temporal signal samples previously required for forming an “ensemble length” along a transmission beam.

It is a further object of the invention to provide such an ultrasound imaging system comprising filtering means to reject the clutter information.

It is a further object of the invention to provide such an ultrasound imaging system for forming either 2-D or 3-D ultrasound Doppler images in real time, such as Doppler flow velocity images or Doppler power images in real time.

The technical problem lies in that, when using such a small number of ensemble lengths (three or four ensemble lengths), it is no more possible to process the ultrasonic image data using third order filters for clutter demodulation. Only second order filters can be used. Such second order filters are less efficient than the known third order filters. They show selectivity proprieties that are drastically reduced with respect to third order filters.

According to the invention, the use of a minimized number of temporal signal samples is compensated by the use of an increased number of spatial signal samples. The spatial information is used several times: in a first stage to perform an adaptive clutter demodulation for estimating flow amplitude to the exception of flow velocity, and in a second stage, to perform a mean clutter demodulation for estimating flow velocity to the exception of flow amplitude.

The ultrasound viewing system of the invention comprises means, hereafter called “small ensemble length filtering”, appropriate to reduce the number of the successive temporal signal samples practically to three or four successive temporal signals required for forming an “ensemble length” along a transmission beam, while having means for clutter filtering. This system presents the advantage of reducing the acquisition time, possibly dividing the acquisition time by more than two. Minimizing the acquisition time duration with respect to the known systems permits of displaying 2-D Doppler images in real time or 3-D Doppler images in real time. The system of the invention presents the further advantage to provide Doppler images that are not deteriorated by the “small ensemble length technique”.

### BRIEF DESCRIPTION OF THE DRAWINGS

The invention is described hereafter in detail in reference to the following diagrammatic drawings, wherein:

FIG. 1 represents a general block diagram of the small ensemble length clutter filtering stage constructed in accordance with the principles of the present invention;

FIG. 2 represents a detailed block diagram of an ultrasonic imaging system constructed in accordance with the principles of the present invention;

FIG. 3 shows a block diagram of an ultrasound apparatus comprising the system of FIG. 1 and FIG. 2.

### DESCRIPTION OF EMBODIMENTS

The invention relates to an ultrasound imaging system, also called ultrasonic viewing system, which has means to form real time 2-D or 3-D Doppler images of fluid flow, for instance the blood flow of a vessel or the heart. This ultrasound viewing system has means to minimize the acquisition time duration with respect to conventional ultrasound systems. According to the invention, only three or four, and no more than five successive signal samples, out of the eight signal samples usually used in the cited prior art, are necessary to measure fluid flow characteristics in a moving body part. This operation of minimizing the number of successive temporal signal samples may divide the acquisition time by two.

According to the invention, the fact that few temporal samples are available is compensated by the fact that a large number of spatial samples is used. The number of temporal signal samples is defined as “ensemble length”, denoted by EL. An object of interest, such as a vessel or the heart, called target, receives three to five successive transmissions pulses, which allows analyzing the temporal variations of the successive signal samples, due to the displacement of the target.

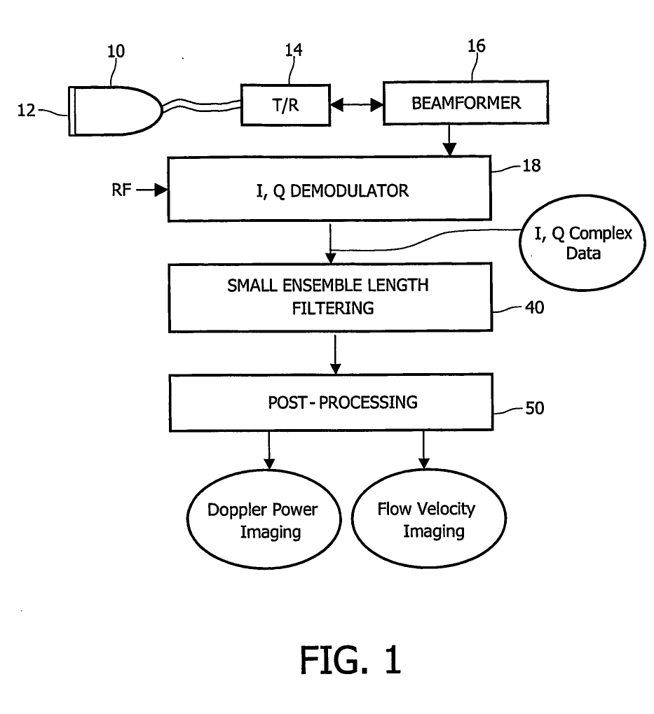

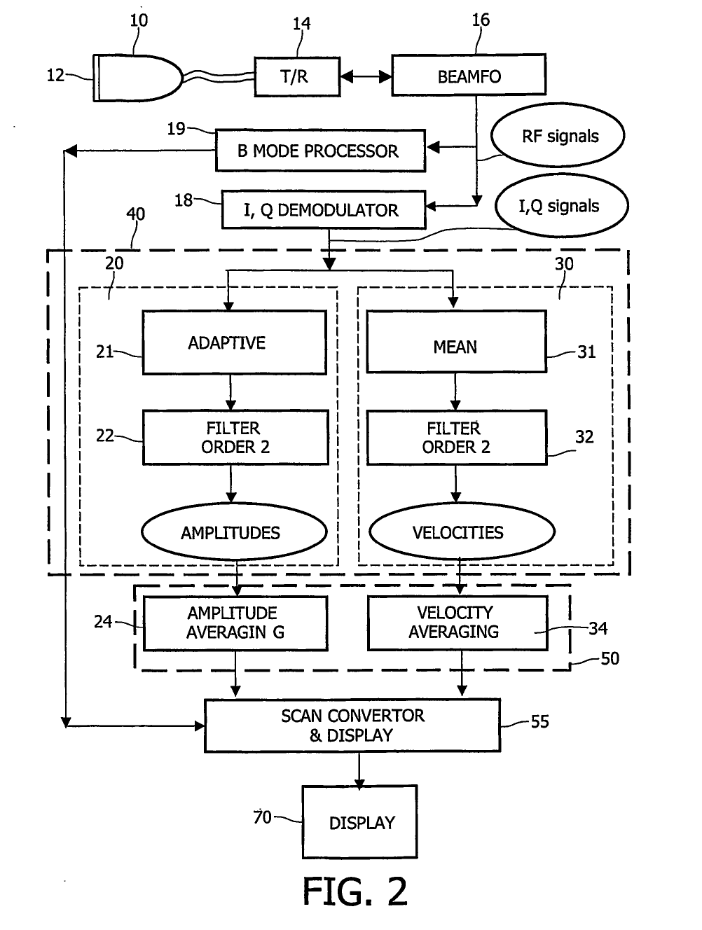

Referring first to FIG. 1, a block diagram of an ultrasonic imaging system constructed in accordance with the principles of the present invention is shown. An ultrasonic probe 10 includes an array of transducer elements 12, which transmits waves of ultrasonic energy into the body of a patient and receives ultrasonic echoes returning from structures in the body. Preferably the probe comprises a 2-D phased array of transducer elements. In the case of ultrasonic wave transmission for Doppler interrogation of the body, the echoes returning from blood and other fluids in the body are of interest. The ultrasonic probe 10 is connected to a transmitter/receiver 14, which alternately pulses individual elements of the transducer to shape and steer an ultrasonic beam, and receives, amplifies and digitizes echo signals received by the transducer elements following each pulse transmission. The transmitter/receiver 14 is coupled to a beamformer 16, which controls the times of activation of specific elements of the transducer 12 by the transmitter/receiver 14. Circuits that are not represented, for performing transmitter/receiver functions and beamformer function, may be partially in the probe and partially outside the probe, thus forming a beamforming system 10, 12, 14, 16 in the imaging system. The timing of activation enables the transducer 12 to transmit a shaped and focused ultrasound beam in a desired direction. The beamformer 16 also receives the digitized echo signals produced by the transmitter/ receiver during echo reception and appropriately delays and sums them to form coherent echo signals. The echo signals produced by the beamformer 16 are coupled to a B mode processor 19, as shown in FIG. 2, and to the I,Q demodulator 18.

According to the invention, for each transmission beam, this beamforming system 10, 12, 14, 16 simultaneously forms, in 2-D or in 3-D, several receive echo signals denoted by RF.

Referring to FIG. 2, the B mode processor 19 processes the amplitude information of the echo signals, denoted by RF, on a spatial basis for the formation of structural images of the tissue in the area of the patient being scanned. The I,Q demodulator 18 demodulates the received echo signals into quadratic components, i. e. complex data, denoted by I,Q, for Doppler processing.

Referring to FIG. 1, according to the invention, the I,Q quadratic complex data, issued by the I,Q demodulator 18 are processed by demodulation means 40, separately applied on velocity data and on amplitude data, which can respectively yield flow velocity data or power data for imaging. The demodulation means 40 processes the I,Q complex data in such a way that the resulting 2-D or 3-D Doppler flow data are produced in real time.

Referring to FIG. 2, for producing flow velocity images, the flow estimation processor 40 is applied to the I, Q demodulated signals and comprises two stages, respectively 20 and 30, for using several times the spatial information, which permits of compensating the use of a minimized number of temporal signal samples:
The first stage 20 has means 21 for performing a step of adaptive clutter demodulation. The adaptive clutter demodulation is used for estimating uniquely the amplitude data, called amplitudes, and not the phase data, called velocities. A reason is that the adaptive clutter demodulation, if applied as described in the cited prior art by TORP et alii, yields distortions on the velocity data. Instead, the adaptive clutter demodulation means 21 is highly appropriate to be applied to the amplitude data. The adaptive clutter demodulation is performed on the spatial information of the signals. When applied purely on the amplitudes, the adaptive clutter demodulation presents the supplementary advantage to avoid the drawbacks due to flash effects, i. e. the effects of acceleration of the clutter.
The second stage 30 has means 31 for performing a step of mean clutter demodulation. The mean clutter demodulation is applied uniquely to the phase data, called velocities, and not the amplitude data. The mean clutter demodulation is performed by temporally averaging the local velocities. This provides a mean estimation of the local clutter velocity, for demodulation, in order to estimating the flow velocity.
The first and the second stages 20, 30 have respective high-pass filtering means 22 and 32 constituted preferably by second order filters. As the number of ensemble lengths is small, third order filters may not be used. Instead, second order filters are quite appropriate. However, these second order filters, respectively 22 and 32, are less efficient that are third order filters, which third order filters may be used in the case when a large number of ensemble lengths is used.

More specifically, the amplitude and phase of the blood flow are evaluated with ensemble lengths of three and four, using high-pass filtering techniques after clutter demodulation. These high-pass filters provide more than 60 dB attenuation at DC, and they have a cutoff that is high enough to eliminate the clutter signal.

The used filters are preferably Infinite Impulse Response (HR) filters. Finite Impulse Response (FIR) filters have the characteristic that they do not introduce distortions on the velocity estimation, however they do not provide sufficient attenuation at DC. Instead, Infinite Impulse Response (IIR) filters provide higher attenuation at DC and have higher slopes than FIR filters but their minimum possible cutoffs are limited mainly due to the small size of the ensemble length.

One main constraint in the definition of such filters is the order limitation related to the small size of the ensemble lengths. The determination of the amplitude of the flow requires at least one valid output sample after filtering, while the determination of the flow velocity using the autocorrelation method requires at least two valid output samples after filtering. With ensemble lengths of four, the filters that can be used are of second order if the flow velocity is required and can be of third order, with some limitations on the obtainable cutoff, if only the flow amplitude is required.

According to the invention, for example, second order filters, denoted by order 2 Butterworth, using a projection initialization method, provide the most complete answer to the previous requirements. Chebyshev-II filters, Rs=−60 dB order 2 filters have similar characteristics. As initialization method, those skilled in the art may use for example a method described by Edward S. Chornoboy, in a publication entitled “Initialization for Improved IIR Filter Performance”, published in IEEE TRANSACTIONS ON SIGNAL PROCESSING, VOL. 40, No. 3, March 1992, or any method yielding appropriate initialization parameters and steps.

In order to evaluate the impact of the EL reduction, the performances of such filters are compared to filters having the same cutoff characteristics but designed for ensemble lengths of six. One main result of the comparison is that the selectivity of filters usable for EL=4 is much lower than for filters usable for EL=6. Indeed, for EL=6, order 3 filters with slopes of 60 dB per decade in the stopband can be used whereas for EL=4, the maximum slope is 40 dB per decade.

The filters amplitude and phase responses are evaluated using complex sinusoidal input data.

The equation characterizing IIR filters is recursive, hence the initialization of such filters is essential. The main methods for initializing the filters are zero, step and projection. Projection initialization is an appropriate approach for ensemble lengths of six or more. Projection initialization is also proposed by Torp et alii, and by E.Chormonoy. It has been verified that projection initialization is still a very appropriate method for ensemble lengths of four. As an example, the projection initialization method of the IIR filters has been successfully applied to the small ensemble lengths technique of the invention.

Hence, referring to FIG. 1 and FIG. 2, the first and the second stages both comprise a post processing stage 50. The post processing stage 50 comprises amplitude averaging means 24 applied to the amplitude data and velocity averaging means 34 applied to the velocity data resulting of the small ensemble length processing means 40. The averaging means 24 and 34 respectively perform a spatial averaging of the results provided by the second order filters 22 and 32 of the respective amplitude data and velocity data. The averaging means 24 and 34, which are applied to spatial information, permit of compensating for the decrease of efficiency of the second order filters that are used in the two stages 20, 30.

It seems that such a spatial averaging would result in a decrease of the image resolution. However, in fact, this spatial averaging operation enhances the structures of interest, which improve the visualization of said object of interest, and eventually improves the visualization of the images.

Referring to FIG. 2, the resulting Doppler data and B-mode data are yielded to a scan convertor and display processor 55 in order to form velocity color flow images and/or power Doppler images, in combination or not with B-mode images. The resulting images are displayed on the display means 70.

Usually, the Doppler flow values are mapped to color values for display. The color values are applied to the scan converter and display processor 55, which spatially arranges the color values in the desired image format. The color values are displayed as pixels on a display 70, wherein each color represents a particular velocity of flow in a particular direction at that pixel location. The color flow velocity information can be overlaid with a structural image of the interior of the body utilizing the structural information provided by the 2-D or 3-D B mode processor 19. This 2-D or 3-D compound color image can show both the direction and velocity of blood flow, as well as the structure of the vessels or organs, which contain the flowing blood.

The Doppler system of FIG. 2 can also display power Doppler images The Doppler power estimates are mapped to display intensity or color values by a color power processor as for color flow velocity data. The 2-D or 3-D Doppler power images may then be displayed on a display 70 or stored in a memory (not represented) and further recalled from the image sequence memory for 2-D or 3-D processing using a peak detector (not represented) for maximum Doppler power intensity detection.

User operation of the system of FIG. 2 is effected through various user controls 65 which enable the user to select the type of imaging to be performed, i e. B mode, Doppler color flow velocity imaging or Doppler color power imaging, and to store and retrieve images from the image sequence memory 64 for three dimensional display, for example.

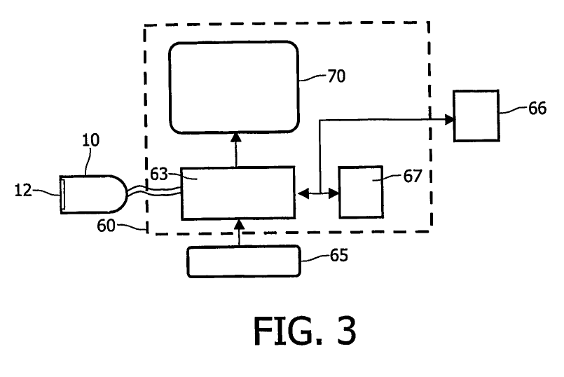

FIG. 3 shows a diagram of an ultrasound examination apparatus according to the invention that is coupled the system of FIG. 1, detailed in FIG. 2. The apparatus comprises a probe 10 for acquiring digital image data of a sequence of images, and ultrasound means 60 for processing these data according to the invention. In particular, the data processing device 60 has computing means 63 and memory means to perform the calculations and construct the images as described above. A computer program product having pre-programmed instructions to carry out the calculations and construct the images may also be implemented. The ultrasound computing means can be applied on stored medical images, for example for estimating medical parameters. The system provides the processed image data to display means and/or storage means. The display means 70 may be a screen. The storage means may be a memory of the system 63. Said storage means may be alternately external storage means. This image viewing system 60 may comprise a suitably programmed computer, or a special purpose processor having circuit means such as LUTs, Memories, Filters, Logic Operators, that are arranged to perform the calculations according to the invention. The system 60 may also comprise a keyboard 65 and a mouse 67. Icones may be provided on the screen to be activated by mouse-clicks, or special pushbuttons may be provided on the system, to constitute control means 66 for the user to actuate the processing means of the system at chosen stages of the calculations. This medical viewing system 60 may be incorporated in an ultrasound examination apparatus. This medical examination apparatus may include a bed on which the patient lies or another element for localizing the patient relative to the apparatus. The image data produced by the ultrasound examination apparatus is fed to the medical viewing system 60. The ultrasound system may be of the mobile kind, to be moved on a trolley.

In the claims:

1. An ultrasound imaging system comprising:
a probe with an array of transducer elements for acquiring ultrasound data of a body, including moving tissue and fluid flow;
a beamforming system for emitting and receiving ultrasound beams in said body, which uses, for each transmission beam, an ensemble length of more than two temporal samples and less than eight;
processing means to process flow Doppler signals comprising adaptive clutter demodulation applied on amplitude signals and means clutter demodulation applied on phase signals followed by high-pass filtering;
and display means to display images based on said processed flow Doppler signals.

2. The ultrasound imaging system of claim 1, wherein the filtering means comprises a second order Infinite Impulse Response filters.

3. The ultrasound imaging system of claim 1, wherein the filtering means comprises a third order Infinite Impulse Response filters for filtering amplitude signals.

4. The ultrasound imaging system of one of claim 1, wherein the filtering means are followed by post-processing means comprising, spatial averaging means for yielding respectively power Doppler signals from the amplitude signals and flow velocity signals from the phase signals.

5. The ultrasound imaging system of claim 1, wherein the post processing means comprises amplitude averaging means applied to the amplitude data and velocity averaging means applied to the velocity data resulting of high-pass filtering means, for performing a spatial averaging of the results provided by said filters and permitting of compensating for the use of small ensemble length less than eight.

6. The ultrasound imaging system of claim 5, wherein the processing means comprises a color flow velocity processor for mapping flow velocity values on color values.

7. The ultrasound phased array imaging system of claim 5, wherein the processing means comprises a color power processor for mapping the estimated power magnitude on color values.

8. The ultrasound imaging system of claim 1, further comprising a B mode processor for processing the amplitude information of the echo signals, on a spatial basis, for the formation of structural images of the tissue.

9. The ultrasound imaging system of claim 1, further comprising:
a display processor for processing the B mode data, color flow velocity data, color power data, and an image memory for memorizing the image data for display; and
a user control for the user to select the images to display in one mode or in combined modes.

10.-11. (canceled)
